(19) United States
(12) Patent Application Publication
(10) Pub. No.: US 2025/0331962 A1
(43) Pub. Date: Oct. 30, 2025

(54) Dental screwdriver

(71) Applicant: SIRIANI TECHNOLOGIES LTD., Kiryat-Ata (IL)

(72) Inventor: Gaby Siriani, Tel Aviv (IL)

(21) Appl. No.: 18/866,537

(22) PCT Filed: May 10, 2023
(86) PCT No.: PCT/IL2023/050479

§ 371 Date: Nov. 17, 2024

(30) Foreign Application Priority Data
May 30, 2022 (IL) 293457

(51) Int. Cl. A61C 8/00

(52) U.S. Cl. CPC A61C 8/0089

(57) ABSTRACT

Dental screwdriver comprising a shank, a screwdriver head, a handle, and a rotatable gear. Shank is aligned longitudinally along a shank-axis. Screwdriver head is disposed at shank distal end along shank-axis and engages with a screw to be tightened or loosened at a jaw location inside mouth of patient. Handle is coupled to shank and has first handle portion aligned longitudinally along a handle-axis orthogonal to shank-axis. Handle is held by first hand of operator. Gear is radially rotatable about shank-axis and coupled to shank proximal end such that shank extends axially from gear central opening. Gear provides torque to drive radial rotation of shank and screwdriver head about shank-axis for tightening or loosening screw. Gear comprises disc or wheel element configured to be rotated using at least one finger of second hand of operator, such that gear, shank and screwdriver head, is rotatable as a single drive unit.

### FIELD OF THE INVENTION

The present invention relates to the field of dental tools and instruments, and in particular to a dental screwdriver for securing or removing screws for dental implants, abutments or prosthetics.

### BACKGROUND OF THE INVENTION

A dental implant is a structure that is inserted into the jawbone of a patient to support a dental prosthetic, such as an artificial tooth (i.e., a crown), a bridge, or a denture, or to act as an orthodontic anchor, such as for dental braces. The dental implant is positioned and secured into the jawbone and usually undergoes an osseointegration process before the dental prosthetic is inserted. Securing of the dental implant to the jawbone, and/or securing of the dental prosthetic to the dental implant, is generally implemented using a screw fastener, which is tightened and loosened manually by means of a dedicated tool, commonly referred to as a dental screwdriver or dental wrench. There are many types of dental screwdrivers having various sizes, shapes and configurations. Some may be designated for particular screw types or for specific procedures, while others may be adaptable for multiple uses and procedures. A dental screwdriver may be integrated with a ratchet device to provide the requisite torque to facilitate the screwing or unscrewing process. However, certain dental screwdriver tools may be cumbersome and difficult to manipulate, and may require extensive training and experience to properly operate.

In certain cases, a dental practitioner may prefer to rely on dexterity and manual sensitivity in order to govern the degree of tightening/loosening during the screwing/unscrewing process, while limiting usage of a ratchet or other torque device only for the final tightening phase or initial loosening phase. However, when initially fastening a screw, or just before completing to unfasten a screw before its removal from the mouth, the screw is able to rotate freely about the insert with minimal resistance. Consequently, the torque force is greater than that of the screw rotation, so that the screwing (or unscrewing) process cannot be completed using a ratchet or torque device but must be done manually or using a smaller screwdriver-head. Moreover, the screwdriver-head (without the handle), and/or the screw and abutment, is liable to fall inside the mouth of the patient, which presents a dangerous safety hazard as the patient may inadvertently swallow these small components and can even lead to choking. This potential hazard may be unavoidable even if the dental practitioner is highly skilled and experienced. One approach for circumventing this problem is to attach the screwdriver head to a segment of string or wire, allowing the screwdriver head to be pulled out and retrieved if it falls inside the mouth.

Various configurations of screwdriver and wrench type dental tools are known in the art. Some examples are disclosed in: PCT application publication No. WO 2020/137782A1 to Jang Kyung-Sam, entitled: “Dental driver”; Chinese Utility Model No. CN202235725 entitled “Dental implant implanting wrench dedicated for orthodontics”; U.S. Pat. No. 11,246,686 to Jinton entitled: “Screwdriver and screw for medical applications, in particular for dental applications”; U.S. Patent Application No. 2007/0281274 to Schraffran et al entitled: “Dental wrench and method of use thereof”; U.S. Patent Application No. 2017/0100205 to Valvanis entitled: “Dental screwdriver”; Chinese Utility Model No. CN211560433 (U) entitled “Oral dental implant screwdriver”; and U.S. Pat. No. 4,976,617 to Carchidi, entitled: “Dental implant screw driver system”.

U.S. Pat. No. 5,192,823 to Hughes, entitled: “Driver tool and method for implant dentistry”, discloses a hand-operated dental tool for installation of implant abutments and screws. The tool has a knob, manually rotatable externally of a patient mouth on one end of an arm, and a bit, releasably secured on a sprocket, rotatable inside the patient mouth in response to rotation of the knob. A chain connects the bit sprocket to a sprocket attached to the knob, so that the bit rotates about an axis parallel to, but laterally offset by the arm from, the axis of rotation of the knob. A resilient elastomer sleeve is releasably stretched over the abutment or screw and over the bit for initial positioning. Multiple, interchangeable bits are provided to match different sized abutment or screw heads. The bit shank is recessed intermediate cylindrical portions, for receiving a set screw.

U.S. Pat. No. 5,458,603 to Futch, Sr., entitled: “Elongated drive tool for prosthesis in body cavity”, discloses a surgical instrument for insertion and removal of component parts involved in prostheses. The surgical instrument includes: an elongated support, adapted to rotary support at the distal end a removable insertion or removal tool; a control knob, at the proximal end adjacent the handle that is engageable by the fingers or thumb on the hand holding the instrument; and a gear train, extending between a tool bit and the control knob. Alternative driving mechanisms include a teethed belt, a spherical ball and link chain, or a bevelled gear.

U.S. Pat. No. 7,100,476 to Feit, entitled: “Torque wrench for dental implants”, discloses a manually operated and adjustable dental implant torque wrench that employs a screw holding sheath for placing a dental screw in a mouth of a patient. A handle offset to avoid opposite teeth to the work area is provided with a bevel gear and pinion set to translate the turning motion from a straight turning shaft in the handle to a perpendicular screw turning head. An assortment of replaceable snap-fit screw turning heads fits the various types of dental screws. An easy turn T-shaped handle with a pivotable turning knob is controlled by a torque limiting device.

### SUMMARY OF THE INVENTION

In accordance with one aspect of the present invention, there is thus provided a dental screwdriver. The dental screwdriver includes a shank, a screwdriver head, a handle, and a rotatable gear. The shank is aligned longitudinally along a shank axis. The screwdriver head is disposed at a distal end of the shank along the shank axis and configured to engage with a screw to be tightened or loosened at a jaw location inside a mouth of a patient. The handle is coupled to the shank and comprises a first handle portion aligned longitudinally along a handle axis orthogonal to the shank axis, the handle configured to be held by a first hand of an operator. The gear is radially rotatable about the shank axis and coupled to a proximal end of the shank such that the shank extends axially from a central opening of the gear, the gear configured to provide torque to drive a radial rotation of the shank and the screwdriver head about the shank axis, for tightening or loosening the screw, where the gear comprises a disc or wheel shaped element configured to be rotated using at least one finger of a second hand of the operator, such that the gear, the shank and the screwdriver head is rotatable as a single drive unit. The gear may be configured to enable radial rotation using a single finger. The handle may include a second handle portion extending longitudinally from the first handle portion at an angular bend relative to the handle axis, to facilitate maneuvering and orienting of the screwdriver head in the mouth of the patient. The angular bend may be adjustable, whereby a locking screw is configured to be selectively inserted at a hinge of the handle so as to affix the second handle portion at a selected angle relative to the handle axis. The dental screwdriver may further include a stopper, disposed on the shank distally from the gear and the handle, the stopper configured to prevent axial displacement of the handle and the gear. The gear may include a diameter in the range of 15 to 22 millimeters (mm). The gear may include a thickness in the range of 3 to 5 millimeters (mm). The gear may include a material or a tread pattern configured to provide traction. The tread pattern may include a plurality of ridges and grooves extending radially across the circumference of the gear. The handle of the dental screwdriver may be a ratchet handle of a ratchet, configured to rotate the rotatable gear The dental screwdriver may further include a ratchet adapter, configured for removably coupling the ratchet with the shank and the rotatable gear. The ratchet adapter may include at least one clip, configured to couple to the ratchet or ratchet handle, and at least one button, configured to activate the clip to disengage the ratchet or ratchet handle. The screw may be configured to secure or remove a dental structure, such as a dental implant, a dental abutment, and/or a dental prosthetic.

In accordance with another aspect of the present invention, there is thus provided a method for using a dental screwdriver. The method includes the step of providing a dental screwdriver comprising: a shank, aligned longitudinally along a shank axis; a screwdriver head, disposed at a distal end of the shank along the shank axis; a handle, coupled to the shank, the handle comprising a first handle portion aligned longitudinally along a handle axis orthogonal to the shank axis; and a rotatable gear, radially rotatable about the shank axis and coupled to a proximal end of the shank such that the shank extends axially from a central opening of the gear, the gear comprising a disc or wheel shaped element configured to be rotated using at least one finger of a second hand of an operator, such that the gear, the shank and the screwdriver head is rotatable as a single drive unit. The method further includes the step of holding the handle with a first hand of the operator, and directing the screwdriver head to a jaw location inside the mouth of a patient. The method further includes the step of radially rotating the gear using at least one finger of a second hand of the operator, providing torque to drive a radial rotation of the shank and the screwdriver head, for tightening or loosening the screw at the jaw location. Radially rotating the gear may be performed using a single finger. The handle may include a second handle portion extending longitudinally from the first handle portion at an angular bend relative to the handle axis, and the step of holding the handle may comprise holding the second handle portion of the handle for directing the screwdriver head. The step of radially rotating the gear may be implemented using a ratchet, removably couplable with the shank and the rotatable gear via a ratchet adapter. The screw may be configured to secure or remove a dental structure, such as a dental implant, a dental abutment, and/or a dental prosthetic.

### BRIEF DESCRIPTION OF THE DRAWINGS

The present invention will be understood and appreciated more fully from the following detailed description taken in conjunction with the drawings in which:

FIG. 1 is a side view illustration of a dental screwdriver, constructed and operative in accordance with an embodiment of the present invention;

FIG. 2 is a side view illustration of a dental screwdriver with a straight handle, constructed and operative in accordance with another embodiment of the present invention;

FIG. 3 is a perspective view illustration of a ratchet adapter configured to connect a rotatable gear of the dental screwdriver to a ratchet, constructed and operative in accordance with an embodiment of the present invention;

FIG. 4A is a perspective view illustration of the dental screwdriver operating on a lingual surface of a posterior tooth, operative in accordance with an embodiment of the present invention;

FIG. 4B is a first perspective view illustration of the dental screwdriver operating on a lingual surface of an anterior tooth, operative in accordance with an embodiment of the present invention; and

FIG. 4C is a second perspective view illustration of the dental screwdriver operating on a lingual surface of an anterior tooth, operative in accordance with an embodiment of the present invention.

### DETAILED DESCRIPTION OF THE EMBODIMENTS

The present invention overcomes the disadvantages of the prior art by providing a dental screwdriver tool that allows for securing and removing of screw-fastened dental structures, such as implants, abutments or prosthetics, in a controlled and straightforward manner, while providing sufficient torque to implement the fastening and unfastening processes. The dental screwdriver is characterized by a rotatable gear, disposed on an end of the screwdriver shank and adapted to provide requisite torque to facilitate the turning of the screwdriver head in a desired direction. The disclosed dental screwdriver allows an operator to apply manual dexterity and sensitivity of one finger during the initial fastening (or unfastening) phases, followed by a final fastening (or unfastening) using two fingers. In this manner the entire process can be performed using only one or two fingers of one hand (inside the mouth of the patient), freeing up the other hand of the operator to hold and suitably position the screwdriver handle (outside the patient mouth). The wide and flat structure of the rotating gear enables sufficient control and manipulation with only one finger, while the circular diameter provides for sufficient torque force for the final fastening and unfastening stages using only two fingers, one on each side of the gear. The elongated handle can be easily manipulated to position and orient the screwdriver head within the mouth for performing the fastening/unfastening process, while preventing the screwdriver head and/or screw from inadvertently falling inside the mouth. The handle may be bent at a designated angle so as to facilitate screwing of different angled screws within the mouth at difficult to reach areas, such as near or toward the side of the tongue. The handle may be provided in various lengths, which may be adapted to required situations. A ratchet adapter may be coupled to the dental screwdriver for attaching to an external ratchet for implementing the gear rotation.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

It will be understood that when an element is referred to as being “on”, “attached” to, “operatively coupled” to, “operatively linked” to, “operatively engaged” with, “connected” to, “coupled” with, “contacting”, “added to”, etc., another element, it can be directly on, attached to, connected to, operatively coupled to, operatively engaged with, coupled with, added to, and/or contacting the other element or intervening elements can also be present. In contrast, when an element is referred to as being “directly contacting” another element or “directly added” to another element, there are no intervening elements and/or steps present.

Whenever the terms “about” or “approximately” are used, it is meant to refer to a measurable value such as an amount, a temporal duration, and the like, and is meant to encompass variations from the specified value, as such variations are appropriate to perform the disclosed methods.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Whenever terms “plurality” and “a plurality” are used it is meant to include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

This disclosure employs open-ended permissive language, indicating for example, that some embodiments “may” employ, involve, or include specific features. The use of the term “may” and other open-ended terminology is intended to indicate that although not every embodiment may employ the specific disclosed feature, at least one embodiment employs the specific disclosed feature.

The terms “user” and “operator” are used interchangeably herein to refer to any individual person or group of persons using or operating a device or method according to one or more embodiments of the present invention, such as a dental practitioner (e.g., dentist, orthodontist, oral surgeon, dental assistant, and the like) performing a dental procedure.

The terms “subject” and “patient” are used interchangeably herein to refer to an individual upon which a device or method according to one or more embodiments of the present invention is operated upon, such as a person upon which a dental procedure (e.g., a dental implant fastening procedure) is performed. The subject may be any living entity, such as a human, animal, or other vertebrate, characterized with one or more teeth on which a dental procedure may be performed.

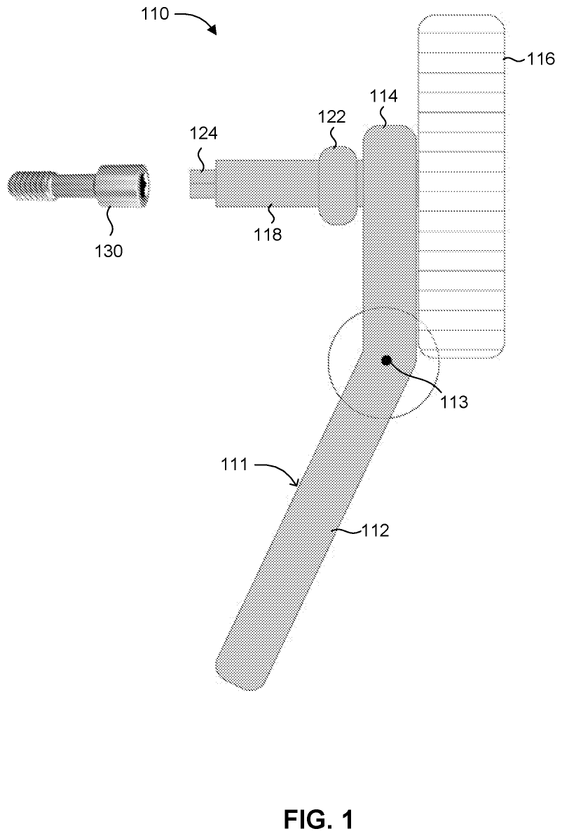

Reference is now made to FIG. 1, which is a side view illustration of a dental screwdriver, generally referenced 110, constructed and operative in accordance with an embodiment of the present invention. Dental screwdriver 110 includes a handle 111, a rotatable gear 116, a shank 118, and a screwdriver head 124. Handle 111 includes a first (e.g., upper) handle portion 114 and an elongated second (e.g., lower) handle portion 112, joined at a handle hinge joint 113. Dental screwdriver 110 may be characterized by a distal end and a proximal end, where the proximal end is closest to an operator (not shown) of dental screwdriver 110, and the distal end faces away from the operator. A “shank axis” is defined herein as an axial direction parallel to the longitudinal axis of shank 118, i.e., towards or away from the proximal end or distal end of dental screwdriver 100. A “handle axis” is defined herein as an axial direction orthogonal to the shank axis, and parallel to the longitudinal axis of upper handle portion 114.

Handle 111 includes a lower handle portion 112 by which an operator (not shown) can hold onto or grip dental screwdriver 110 using one hand. Shank 118 extends axially through an opening of upper handle portion 114. Screwdriver head 124 is positioned at the distal tip of shank 118, and is adapted to engage with a screw head of a dental screw 130 to be fastened (tightened) or unfastened (loosened) within the mouth of the subject. Screwdriver head 124 may be integrally formed with shank 118, which can help prevent screwdriver head 124 from detaching and inadvertently falling inside the patient mouth when manipulating the dental screw. Alternatively, screwdriver head 124 may be a separate add-on element configured to be selectively coupled or attached to the distal end of shank 118, such as to match a particular size or shape of screw head. Screw 130 is depicted in FIG. 1 for illustrative purposes as having a hexagonal head, but may generally consist of other screw head sizes and shapes, and may have any thread length, as applicable for fastening the requisite dental structure (e.g., a dental implant, abutment or prosthetic). Shank 118 may be any suitable length and diameter.

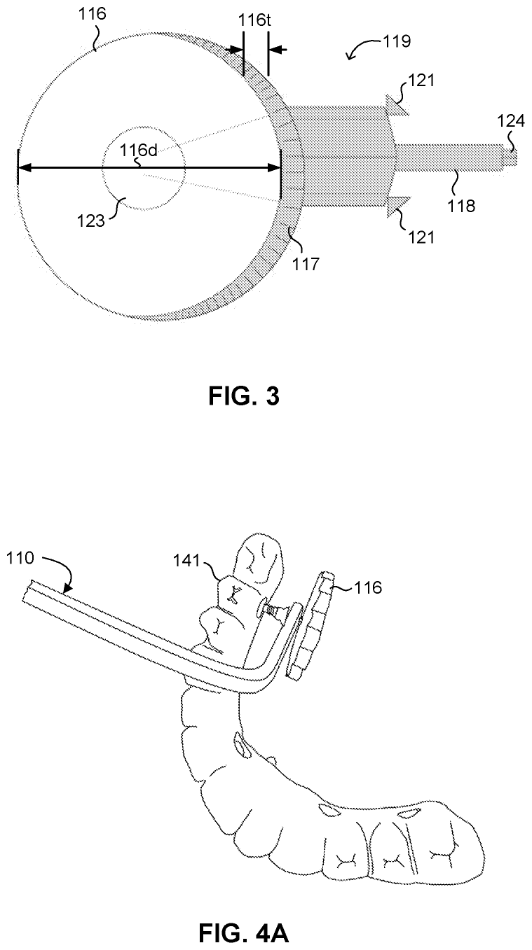

Rotatable gear 116 is a circular disc or wheel shaped element positioned at a proximal end of shank 118, proximal from upper handle portion 114. Shank 118 extends axially and is fixedly coupled in a central opening of gear 116, such that gear 116, shank 118 and screwdriver head 124 are radially rotatable about the shank axis as a single drive unit. Gear 116 is configured to be rotated using a single finger of the operator, e.g., an index finger, so as to provide torque or counterforce when operating screwdriver 110. For example, turning gear 116 in a first radial direction (e.g., clockwise) may provide requisite torque for tightening a screw 130, while turning gear 116 in an opposite radial direction (e.g., counterclockwise) may provide requisite torque for loosening a screw 130. The surface of gear 116 may be characterized by a material and/or a tread pattern adapted to provide enhanced traction so as to facilitate rotation. For example, gear surface 117 of gear 116 (shown in FIG. 3) may have a series of grooves and ridges extending radially across the circumference thereof, such as resembling a tread pattern of a vehicle tire, to provide greater friction to assist the turning of gear 116, to enhance grip, and/or to prevent the finger from slipping off the gear surface 117.

An optional stopper 122 is disposed on and around shank 118 distally from upper handle portion 114 and gear 116. Stopper 122 may be embodied by a small fragment of plastic, rubber or metal, and configured to prevent axial displacement of upper handle portion 114 and of gear 116 so as to maintain these components securely positioned on shank 118.

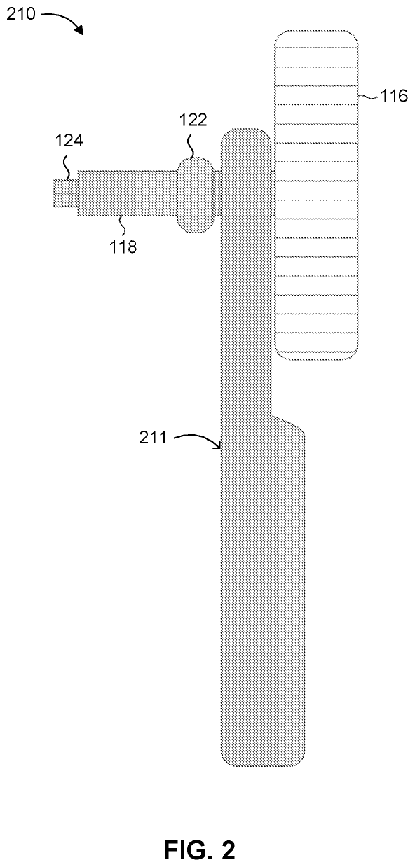

Lower handle portion 112 of handle 111 may be formed at a fixed angle relative to upper handle portion 114. Alternatively, lower handle portion 112 may be selectively bent or pivoted about handle hinge 113 to a desired orientation, providing a contoured handle shape to enable easier manipulation of dental screwdriver 110 by the operator and to facilitate access to difficult to reach areas within the subject mouth. For example, handle hinge 113 may include a locking screw at a pivot point, which can be removed to allow manually orienting lower handle portion 112 to a desired angle, and then reinserted to lock lower handle portion 112 in place at the desired angle. Further alternatively, handle 111 may be straight without a bend, such that the upper handle portion 114 and lower handle portion 112 form a single integral unit, which may thus be more stable with fewer moving parts. Reference is made to FIG. 2, which is an illustration of a dental screwdriver, referenced 210, with a straight handle 211. Dental screwdriver 210 is analogous to dental screwdriver 110 except that handle 211 is straight rather than bent at a fixed or adjustable angle about a hinge point.

According to an embodiment, rotatable gear 116 may be rotated by means of a ratchet, similar to standard ratchet tools configured to enable continuous rotary motion in only one direction while preventing motion in an opposite direction. The ratchet may be coupled to rotatable gear 116 using a suitable coupling mechanism and may supplement or replace selected elements of dental screwdriver 110, such as handle 111. In particular, an external ratchet (i.e., separate from dental screwdriver 110) may be connected to shank 118 via an adapter, so as to enable rotation of gear 116 by holding and manipulating a ratchet handle of the ratchet. Reference is made to FIG. 3, which is a perspective view illustration of a ratchet adapter, referenced 119, configured to connect a rotatable gear of the dental screwdriver to a ratchet, constructed and operative in accordance with an embodiment of the present invention. Ratchet adapter 119 may have a socket (female connector) or a plug (male connector), such as hexagonal-shaped or star-shaped connector, and be adapted to fit a standard ratchet tool (not shown), to enable the operator to utilize an external ratchet for implementing the rotation of rotatable gear 116. Accordingly, shank 118 is axially coupled at the distal end of ratchet adapter 119 and extends axially to rotatable gear 116 at the proximal end of ratchet adapter 119, such that shank 118, gear 116 and screwdriver head 124 are radially rotatable as a single unit upon the turning of gear 116. The operator can then implement the gear rotation by holding the ratchet handle (not shown) and repetitively turning the handle in an appropriate direction (e.g., clockwise and/or counterclockwise). Ratchet adapter 119 may include a spring-loaded locking mechanism for securely attaching and removing the ratchet (or ratchet handle). For example, ratchet adapter 119 may include one or more clips 121 for securely fastening a ratchet. Clips 121 may be activated by pressing a disengagement button 123 on the middle of gear 116 to release the locking mechanism and allow for removal of the ratchet. It is noted that a basic ratchet may be coupled to shank 118, gear 116 and screwdriver head 124 of dental screwdriver 110 (i.e., to be used instead of handle 111), or alternatively a ratchet with an integrated screwdriver head may be coupled to only shank 116 and gear 118 (i.e., to be used in replacement of both screwdriver head 124 and handle 111). Consequently, a ratchet adapter may not be applicable when the screwdriver head 124 is integrally fixed to shank 118 and handle 111, 211, as in the embodiments depicted in FIGS. 1 and 2.

Dental screwdriver 110 may optionally include and/or be associated with additional components not shown in the Figures for enabling the implementation of the disclosed subject matter. For example, dental screwdriver 110 may include a shield or protective covering configured to cover the distal end of shank 116 and screwdriver head 124 when dental screwdriver 110 is in storage and not in use, and to be removed before operation, so as to minimize wear and tear and to protect against inadvertent injury from an exposed screwdriver head.

To operate dental screwdriver 110 for fastening/tightening or unfastening/loosening of a dental screw 130, the operator (e.g., dental practitioner) holds dental screwdriver 110 with one hand (e.g., using his/her dominant hand) at the lower handle portion 112 of handle 111, and directs the screwdriver head 124 in a distal direction toward the mouth of the patient. The operator then engages the screwdriver head 124 of dental screwdriver 110 with the screw head of dental screw 130, which has been positioned within the mouth of the patient, e.g., at a jaw location where a dental implant, abutment or prosthetic is to be secured or removed. Once the screwdriver head 124 is properly engaged, the operator can turn rotatable gear 116 with one finger of a second hand (e.g., using his/her non-dominant hand) substantially in the subject mouth, to provide torque for turning the screw head to tighten or loosen screw 130, as needed. The operator may sense manually when the screw 130 has been sufficiently tightened or loosened, such as when the turning finger encounters significant resistance during the turning process, to provide an indication for when to cease the tightening or loosening process. The operator may utilize manual sensitivity of one finger during the initial tightening (or loosening) phases and then complete the process using two fingers. After the dental screw 130 has been fully tightened or loosened, the operator (or other dental practitioner) may continue to perform the remainder of the dental procedure. For example, the tightening or loosing of the dental screw 130 may be a requisite initial stage in the installation or removal of a dental implant, the installation or removal of a dental prosthesis (e.g., artificial tooth) onto or from an implant or abutment, the installation or removal of an orthodontic appliance (e.g., braces), and the like.

The dimensions of rotatable gear 116 is selected so as to facilitate rotation, while still providing sufficient torque when tightening or loosening a screw. For example, the diameter 116d of gear 116 (depicted in FIG. 3) may be in the range of approximately 15 millimeters (mm) to 22 millimeters (mm), and the thickness 116t of gear 116 (depicted in FIG. 3) may be in the range of approximately 3 mm to 5 mm.

Similarly, the dimensions of handle 111, 211 may be selected so as to facilitate usage and maneuvering of dental screwdriver 110. For example, the upper handle portion 114 (i.e., the portion of handle 111 from shank 118 to the handle hinge joint 113) may be characterized by a length between approximately 2 centimeters (cm) to 2.5 centimeters (cm). The handle may be formed at various lengths to optimize usage.

According to an aspect of the present invention, a method for using a dental screwdriver for tightening or loosening a dental screw, such as to secure or remove a dental implant, abutment or prosthetic, may be provided. The method may include: providing a dental screwdriver including at least: a shank, aligned longitudinally along a shank axis; a screwdriver head, disposed at a distal end of a shank along the shank axis; a handle, coupled to the shank, and comprising a first handle portion aligned longitudinally along a handle axis orthogonal to the shank axis; and a rotatable gear, radially rotatable about the shank axis and coupled to a proximal end of the shank such that the shank extends axially from a central opening of the gear, where the gear comprises a disc or wheel shaped element configured to be rotated using at least one finger of a second hand of the operator, such that the gear, the shank and the screwdriver head is rotatable as a single drive unit. The method further includes holding the handle of the dental screwdriver with a first hand, and directing the screwdriver head to a jaw location inside the mouth of the patient. The method further includes radially rotating the gear using at least one finger of a second hand, providing torque to drive a radial rotation of the shank and the screwdriver head, for tightening or loosening the screw. The gear may be radially rotated using a single finger during an initial tightening or loosening phase, and radially rotated using two fingers (i.e., on opposite sides of the gear) during a final tightening or loosening phase. After the screw has been fully tightened or loosened, the remainder of the dental procedure may be continued.

It is appreciated that the disclosed dental screwdriver may allow for tightening or loosening a dental screw using only one finger of one hand (inside the mouth of the patient), freeing up the other hand to hold and properly position the screwdriver handle (outside the patient mouth). The wide and flat structure of the rotating gear may enable sufficient control and manipulation with only one finger, while the circular diameter may provide for sufficient torque force for the final tightening or loosening stages using only two fingers, one on each side of the gear. The operator is able to feel the resistance of the screwdriver head while rotating the gear and may regulate the level of applied torque in accordance with the felt resistance. The advancement of the dental screw may be precisely sensed by the operator manually, allowing for monitoring proper implementation of the dental procedure. For example, the operator can ascertain that a dental implant is securely mounted by perceiving whether the screw penetrates smoothly within the implant or whether there is an obstruction or impediment hindering the insertion (or removal) of the screw.

Furthermore, the elongated handle can be easily manipulated to position and orient the screwdriver head within the mouth for performing the fastening/unfastening, while preventing the screw, the dental structure (e.g., abutment) and/or the screwdriver head from inadvertently falling inside the mouth. The handle may be fixedly or selectively bent at a desired angle so as to facilitate screwing/unscrewing at various angles and positions within the mouth, including difficult to reach areas. In particular, the lower handle portion may be configured or adjusted (e.g., pivoted about the handle hinge) to a selected orientation relative to the upper handle portion, to assist maneuvering the screwdriver head within the patient mouth, and facilitating access to portions of the mouth that are difficult to access, such as for performing lingual procedures (near or toward the side of the tongue).

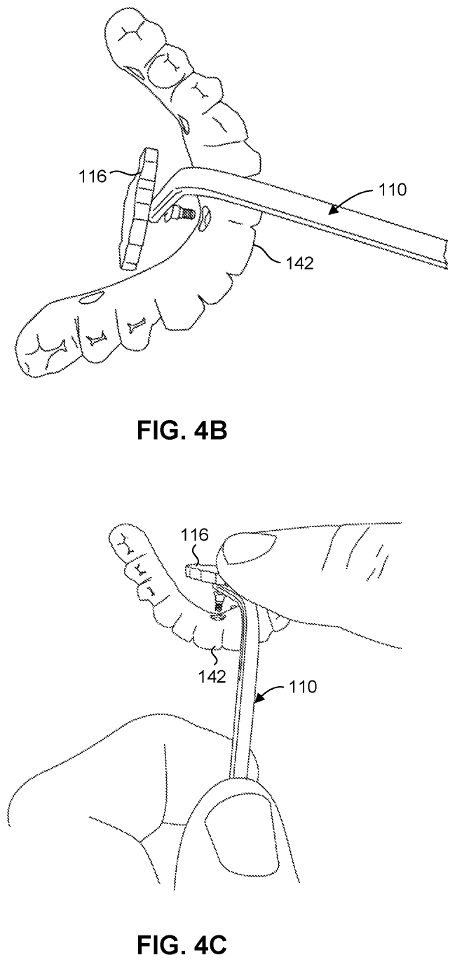

Reference is made to FIGS. 4A, 4B and 4C, respectively directed to perspective view illustrations of the dental screwdriver operating on lingual tooth surfaces, in accordance with embodiments of the present invention. FIG. 4A illustrates the dental screwdriver 110 operating on a lingual surface of a posterior tooth 141, such as a molar. FIG. 4B illustrates the dental screwdriver operating on a lingual surface of an anterior tooth 142, such as an incisor. FIG. 4C illustrates another view of the dental screwdriver operating on a lingual surface of an anterior tooth 142, where the operator is turning rotatable gear 116 with a finger to fasten (or unfasten) the dental screw.

While the disclosed screwdriver is intended primarily for tightening or loosening of dental screws, it may additionally be used for tightening or loosening other types of screws in other applications.

While certain embodiments of the disclosed subject matter have been described, so as to enable one of skill in the art to practice the present invention, the preceding description is intended to be exemplary only. It should not be used to limit the scope of the disclosed subject matter, which should be determined by reference to the following claims.

1. A dental screwdriver, comprising:
a shank, aligned longitudinally along a shank axis;
a screwdriver head, disposed at a distal end of the shank along the shank axis, and configured to engage with a screw to be tightened or loosened at a jaw location inside a mouth of a patient;
a handle, coupled to the shank, the handle comprising a first handle portion aligned longitudinally along a handle axis orthogonal to the shank axis, the handle configured to be held by a first hand of an operator; and
a rotatable gear, radially rotatable about the shank axis and coupled to a proximal end of the shank such that the shank extends axially from a central opening of the gear, the gear configured to provide torque to drive a radial rotation of the shank and the screwdriver head about the shank axis, for tightening or loosening the screw, wherein the gear comprises a disc or wheel shaped element configured to be rotated using at least one finger of a second hand of the operator, such that the gear, the shank and the screwdriver head is rotatable as a single drive unit.

2. The dental screwdriver of claim 1, wherein the gear is configured to enable radial rotation using a single finger.

3. The dental screwdriver of claim 1, wherein the handle comprises a second handle portion extending longitudinally from the first handle portion at an angular bend relative to the handle axis, to facilitate maneuvering and orienting of the screwdriver head in the mouth of the patient.

4. The dental screwdriver of claim 3, wherein the angular bend is adjustable, whereby a locking screw is configured to be selectively inserted at a hinge of the handle so as to affix the second handle portion at a selected angle relative to the handle axis.

5. The dental screwdriver of claim 1, further comprising a stopper, disposed on the shank distally from the gear and the handle, the stopper configured to prevent axial displacement of the handle and the gear.

6. The dental screwdriver of claim 1, wherein the gear comprises a diameter in the range of 15 to 22 millimeters (mm).

7. The dental screwdriver of claim 1, wherein the gear comprises a thickness in the range of 3 to 5 millimeters (mm).

8. The dental screwdriver of claim 1, wherein the gear comprises a material or a tread pattern configured to provide enhanced traction.

9. The dental screwdriver of claim 8, wherein the tread pattern comprises a plurality of ridges and grooves extending radially across the circumference of the gear.

10. The dental screwdriver of claim 1, wherein the handle comprises a ratchet handle of a ratchet, configured to rotate the rotatable gear.

11. The dental screwdriver of claim 10, further comprising a ratchet adapter, configured for removably coupling the ratchet with the shank and the rotatable gear.

12. The dental screwdriver of claim 11, wherein the ratchet adapter comprises:
at least one clip, configured to couple to the ratchet or ratchet handle; and
at least one button, configured to activate the clip to disengage the ratchet or ratchet handle.

13. The dental screwdriver of claim 1, wherein the screw is configured to secure or remove a dental structure selected from the group consisting of: a dental implant; a dental abutment; and a dental prosthetic.

14. A method for using a dental screwdriver, the method comprising the steps of:
providing a dental screwdriver comprising:
a shank, aligned longitudinally along a shank axis;
a screwdriver head, disposed at a distal end of the shank along the shank axis;
a handle, coupled to the shank, the handle comprising a first handle portion aligned longitudinally along a handle axis orthogonal to the shank axis; and
a rotatable gear, radially rotatable about the shank axis and coupled to a proximal end of the shank such that the shank extends axially from a central opening of the gear, the gear comprising a disc or wheel shaped element configured to be rotated using at least one finger of a second hand of an operator, such that the gear, the shank and the screwdriver head is rotatable as a single drive unit;
holding the handle with a first hand of the operator, and directing the screwdriver head to a jaw location inside the mouth of a patient; and
radially rotating the gear using at least one finger of a second hand of the operator, providing torque to drive a radial rotation of the shank and the screwdriver head, for tightening or loosening the screw at the jaw location.

15. The method of claim 14, comprising radially rotating the gear using a single finger of the second hand of the operator.

16. The method of claim 14, wherein the handle comprises a second handle portion extending longitudinally from the first handle portion at an angular bend relative to the handle axis, and wherein the step of holding the handle comprises holding the second handle portion of the handle for directing the screwdriver head.

17. The method of claim 14, wherein the step of radially rotating the gear is implemented using a ratchet, removably couplable with the shank and the rotatable gear via a ratchet adapter.

18. The method of claim 14, wherein the screw is configured to secure or remove a dental structure selected from the group consisting of: a dental implant; a dental abutment; and a dental prosthetic.
